(19) United States
(12) Patent Application Publication
(10) Pub. No.: US 2006/0115429 A1
(43) Pub. Date: Jun. 1, 2006

(54) Biological systems analysis

(76) Inventors: Noubar Afeyan, Lexington, MA (US); Aram Adourian, Woburn, MA (US); Amir A. Handzel, Watertown, MA (US); Brian M. Baynes, Somerville, MA (US)

Correspondence Address: CLARK & ELBING LLP, 101 FEDERAL STREET, BOSTON, MA, 02110, US

(21) Appl. No.: 10/999,512

(22) Filed: Nov. 30, 2004

(51) Int. Cl. A61K 49/00; G06F 19/00; A61B 5/00

(52) U.S. Cl. 424/9.1; 600/300; 702/19

(57) ABSTRACT

Disclosed are methods for the practice of systems pharmacology, systems toxicology, and systems pathology using patterns, such as images, reflective of the biological state of subjects such as humans or experimental mammals. The patterns are generated from data obtained from one or more samples from one or more subjects by applying certain data treatment techniques, and are reflective of the biochemistry of the subjects. The patterns are used in drug selection and discovery, assessment of toxicity and drug efficacy, segmentation of populations, discovery of disease subtypes, as surrogate end points, in the assessment of therapeutic options, and for diagnosis and prognosis of disease.

### BACKGROUND OF THE INVENTION

The invention relates to gaining insights into biological states, e.g., disease states, by gathering biochemical data and manipulating data such that informative patterns emerge. More particularly, the invention provides methods to probe the systems biology of humans and animals so as to enable detection, monitoring, and assessment of the biochemistries which define and characterize various biological states.

### SUMMARY OF THE INVENTION

Simply stated, the invention provides new ways of analyzing complex biochemical information from samples taken from mammals, such as human subjects, and generating molecular systems patterns, including visually striking images, which characterize biological states as diverse as diseased, drug-treated, and even fatigued and stressed. In essence, the invention allows the translation of a phenotype into a complex and highly informative pattern characteristic of the biochemistry of that phenotype.

Many of the molecular systems patterns of the invention can take the form of images, which are easily recognized by the human eye (doctors, clinical researchers) and can be used to distinguish between different biological states, often at a glance. These images and other patterns have a wide range of uses in the medical field. In the practice of medicine, systems pathology employs the patterns of the invention to assess states of health/disease. The patterns may be read by computer, or by eye, in any appropriate setting, such as clinical laboratories or hospitals. In the practice of systems toxicology, drugs or drug candidates are assessed for toxicity, for determination of therapeutic margin, and for short and long-term side effects. In systems pharmacology, the patterns are used by the pharmaceutical industry for assessment of drug efficacy, drug selection, and other properties as discussed herein.

Patterns of the invention provide what is essentially a biochemical snap shot, readable by a computer or the human eye, of a biological state of a subject. These can be used by professionals to assess biochemical states in a way that is analogous to the use of radiological techniques to assess anatomical states.

A molecular systems pattern for an individual is obtained by first using a study set of data from selected subjects to develop a mapping key, and then applying that key to data sampled from individuals so as to discern the biological state of the individuals.

First, multiple individuals are typically selected or recruited to generate data that will serve as a study set. The subjects ideally are phenotype matched individuals of the same species who may be divided into two groups, e.g., diseased (or other biological state under investigation) and control (e.g., healthy, or diseased but successfully drugged). Phenotype matched subjects are, for example, the same sex, close in age and general health, perhaps the same race or ethnicity, and otherwise selected so as to have a personal biochemistry as similar as possible, except with respect to the phenotype of the biological state under study. Samples, e.g., blood, urine, or lymph, are obtained from each subject, with the sample type generally being dictated by the information about the biological state of the mammal being sought. For example, assessment of the toxicity of a drug to kidney cells might drive the choice of urine or kidney tissue biopsy as the sample. One or more samples are taken from each individual in parallel, i.e., all samples taken from the subjects are products of the same sampling protocol. Thus, for example, a study set for development of a molecular systems pattern, e.g., an image, of Alzheimer's disease can be generated from a process that samples same sex septuagenarians on the same diet by sampling blood serum and first in the morning urine.

Next, a multiplicity of biomolecules, e.g., lipids, proteins, peptides, metabolites, and mRNA (frequently tens to hundreds of such biomolecules) are measured, by any appropriate known technique, e.g., mass spectrometry, liquid chromatography, gas chromatography, or nuclear magnetic resonance spectroscopy, various combinations thereof, or techniques hereafter developed. This step yields a large data set indicative of relative concentrations of a large number of biomolecules in each of the multiple study samples. Frequently, a single biomolecule detected by a measurement technique may give rise to a multiplicity of measurement features, such as multiple nuclear magnetic resonance spectroscopy peaks deriving from a single biomolecule, or a multiplicity of molecular fragments derived from a single biomolecule as detected by a particular mass spectrometry system. All, many, or most of the biomolecules or measurement features may not, and need not be, identified. Optionally, but preferably, the data then are filtered to enrich with respect to data which are judged to have some level of involvement, directly or indirectly, with the biological state under study. Thus, the data may be analyzed by statistical methods with the goal of discarding a portion which is static or random across the subject population, or otherwise not likely involved in the biochemistry of the biological state under study. This may be done conveniently with commercially available software. Also optionally, but preferably, the data are normalized so that the concentration of each biomolecule is expressed in a relative and consistent range, e.g., from 0 to 10, or from −1 to +1.

At this point, the data may be arranged in a table with, for example, the subjects identified across the top, and the data from that subject arranged in a column beneath. The data sets for each subject (a column in the illustration), or for each biomolecule, or measurement feature arising from said biomolecule, across the samples (a row) may be expressed in the form of a graph which can be characterized by various mathematical techniques. Next, the data are treated by an algorithm, e.g., an SOM algorithm, in an iterative process to arrange each row of data (or for a pathology map, a column) such that the data for each biomolecule is mapped to a point (pixel, element, or cell), e.g., on a grid, and such that adjacent points, e.g., on the grid, have values as similar as possible. When a satisfactory solution is achieved, the program stores a mapping key or table, i.e., a set of instructions which dictate the location on a grid of each data point in a sample taken from a subject.

At this point, a data set from any one of the study subjects, or a data set created from a new subject, sampled, analyzed, and filtered in a parallel way, when mapped using the mapping key or table, produces a pattern which characterizes the biological state of the individual subject. The pattern may remain as a data structure in a computer and compared with others or recognized as indicative of a particular biological state by a program designed for the purpose.

Alternatively, the pattern can be converted to a visible image which can be recognized by a human as being characteristic of the biological state of the subject from whom the sample was taken. Where it is desired that the pattern be displayed as a visually recognizable image, the data from the individual, which are optionally filtered, are processed by software which specifies the position of each data point in two or three dimensional space, to produce a molecular systems image (MSI). Each point in the image is assigned a color, grayscale, or other means to indicate its value, so as to display a visually recognizable, e.g., colored image.

The information that relates each data point to a position within the image (that is, the mapping key or table), as noted above, preferably is generated by Self Organizing Map (SOM) software or other data treatment software operating on a study set to cluster data based on concentration similarities. Once the data are clustered, applying the mapping key discovered by the program to data from a sample from a new subject, or one of the subjects in the study set, produces a field of abstract shapes in a pattern that can be recognized as being characteristic of a given biological state, e.g., indicative that the subject is in a state of normalcy, toxicity, disease, drugged, etc.

One can compare the content of a pattern, including an MSI from an individual, directly or indirectly to one or more reference patterns. These are generated in the same manner as the test pattern generated from a sample taken from the individual under study. The reference pattern or patterns are produced from the same biomolecules as detected in the test sample and are mapped with the same mapping key. The difference is that, the reference pattern is known by observation to correspond to a particular phenotype. Also a reference pattern may be constructed from a number of subjects known to be in a given biological state, and each data point in the pattern can represent a composite of samples from multiple mammals of the same species.

Within the framework described above, an enormous number of practical, medically-relevant uses of the technology emerge.

One high value use for patterns, e.g., MSI's, is in pharmacology studies. As an example, MSIs of diseased and healthy individuals can be constructed. A drug candidate then is administered to a diseased individual, and an MSI is generated from a sample taken from the individual while under the influence of the drug. This can be compared to the MSI of one or more healthy individuals, a diseased individual treated successfully with a drug, or the MSI of a diseased individual. Comparison of the patterns or images can suggest that the drug candidate might be efficacious, as it might have altered the pattern toward the healthy MSI, or altered the pattern toward the MSI of the successfully drugged individual.

Any drug candidates can be assessed in this manner, including, in particular, known drug substances for which new uses are proposed, and combinations of drugs in which neither, one, or both are known to be efficacious in treating the disease. The drug can also be a new compound which was discovered empirically or designed using a rational drug design method aimed at the disease state.

Another important use of the invention is in assessing toxicity of a substance or combination of substances, usually a drug candidate. In this embodiment, a test mammal, such as a human subject, is administered the drug and a molecular systems pattern is generated from a sample taken from the subject. The test pattern is then compared to one or more reference patterns, which may be generated, for example, from one or more samples from a mammal of the same species to which a known substance toxic to the mammal has been administered, from the same individual mammal before the substance has been administered, from several mammals exhibiting a variety of different toxic responses, or from a mammal administered the substance which is known to tolerate the substance. If, for example, the test pattern resembles the toxic reference pattern, but not the pattern generated from non-drugged healthy mammals, that may be an indicator of the possible toxicity of the drug candidate to the test animal. The comparisons to determine toxicity, as is the case with other determinations according to the invention, can be done by computer, in which no visual image need be generated, or the data can be processed to form and display MSIs, which can be visually compared by a physician or a pharmaceutical research scientist. As is shown in the Figures, differences in MSIs between, for example, animals administered a drug and not administered a drug, are striking, and immediately recognizable by the human eye.

A pathology map is generated in a way similar to the method for creating the mapping key discussed above. But in this case, instead of clustering data characterizing all the biomolecules in a given row, data characterizing all of the biomolecules from each subject (in each column) are clustered. Thus, composite values indicative of the biochemical profile from each individual are grouped by similarity. When the software arrives at a good solution, the resulting pattern is embodied as an array of points, each of which represents an individual sample (and an individual subject). These also can be imaged in the same way as an MSI is imaged. Such maps can be used to reveal subtypes of disease and to group individual subjects based on similarity of their biochemistry, as opposed to just their presenting clinical symptoms. In a pathology map, each data point represents a composite value of the relative concentrations of multiple biomolecules in a sample from a single mammal or group of mammals.

The molecular pathology maps have a variety of powerful utilities. In one embodiment, the maps are used to reveal biochemically distinct forms of apparently similar biological states, e.g., to segment disease into subcategories that may portend different outcomes or indicate different modes of treatment. When a molecular pathology map is generated from data derived from human subjects, all of whom are either healthy or exhibit the same or a similar disease state, and all of whom have been administered the same drug, the map frequently will exhibit a clustering pattern, from which, despite phenotypic similarities among diseased subjects, it becomes immediately apparent that the subjects' physiological and biochemical responses to the drug differ.

Maps can also be used in studies in which patients can be grouped, in advance of the generation of the map, into one which has been observed to respond in one phenotypic manner to the drug, e.g., exhibits a mitigation of the disease, and another which exhibits a different phenotypic response, e.g., no mitigation. On a map produced as disclosed herein from data generated from samples taken from both groups, the observed phenotypic differences appear as clusters of individuals who display biochemical differences. The researcher then can make and compare MSIs of the biological states of individuals within groupings of patients which may permit her to predict in advance of drug administration who will benefit and who will not. If the cells or pixels in the map are linked to the underlying data, the researcher also may be provided a path to discover the biochemical reasons for the differences in response.

Both the molecular systems patterns, including images, and the molecular pathology maps can be used to signal possible side effects of a drug, induced either by a candidate drug to be administered to a human or animal, or induced by an established drug only in a subgroup of patients. To detect possible side effects, a sample from a test subject to whom the drug has been administered is compared to a reference pattern generated from informative samples, e.g., samples from subjects that have been administered the same or a different known drug which in them caused side effects, and/or from subjects to whom drugs have not been administered. This use of the technology finds particular utility in clinical trials, where a potentially useful drug might have side effects in a small portion of the population which is not easily identifiable by conventional techniques. If an individual being considered for enrollment in a trial provides a sample which generates a pattern, e.g., an image, which closely resembles reference images characteristic of side effects for the class of drugs in which the drug candidate belongs, that subject is excluded from the trial. Similarly, individuals can be tested, and their molecular systems patterns compared to reference patterns to identify patients who are likely to suffer side effects from treatment with the drug, are likely to benefit, or are unlikely to benefit.

The methods described herein necessarily involve analysis of data sets from a plurality of individuals of known phenotype or confirmed diagnosis and controls, e.g., healthy individuals, for the purposes of generating an informative study set by clustering biomolecules or subjects according to an algorithm. The data sets may include measurements derived from more than one biological sample type, more than one type of measurement technique, more than one type of biomolecule, or a combination thereof. The subjects of the exercises typically are mammals, such as a human, or a test rodent, canine, or primate. Types of biomolecules include proteins (including post-translationally modified proteins), peptides, nucleic acids (e.g., genes and gene transcripts), and small molecules and metabolites (including lipids, steroids, amino acids, nucleotides, sugars, hormones, organic acids, bile acids, eicosanoids, neuropeptides, vitamins, neurotransmitters, carbohydrates, ionic organics, nucleotides, inorganics, xenobiotics, peptides, trace elements, pharmacophores, and drug breakdown products). Data sets may include measurements from two samples of a single biological sample type that are treated differently, or from one biological sample type that is collected or analyzed at different times. Data sets may also include measurements from different instrument configurations of a single type of measurement technique.

Subsequent to developing a pattern for a biological state, the pattern can be compared to another pattern, where the biological systems being compared are the same or different. A pattern, or combination (either linear or nonlinear) of patterns, can also be compared to a database of patterns to evaluate whether a biological state matches or is similar to a known state.

A “pattern” as used herein is a representation of clustered data representing distinctive features or characteristics of a biological system, e.g., of a mammal such as a human. The data can include measurements or features derived from a biological sample type, a type of measurement technique, and type of biomolecule. The data often are spectral or chromatographic features that are in the form of a graph, table, or some similar data compilation. The pattern may exist only in a computer as a virtual data structure. An exemplary pattern is a two-dimensional image produced by an SOM in which the coordinates correspond to subjects or biomolecules (or features thereof). Other forms of pattern display in addition to two dimensional images may be exploited, e.g., three dimensional displays or radial displays.

A pattern can be considered to include multiple “biomarkers” of a biological system. A biomarker generally refers to a type of biomolecule, e.g., a gene, a gene transcript, a protein or a metabolite, whose qualitative and/or quantitative presence or absence in a biological system is an indicator of a biological state of a mammal. Thus, a pattern can be considered to be a set of biomarkers, e.g., spectral or chromatographic features, that permit in combination characterization of a biological state yet which individually typically are uninformative or only poorly informative. A pattern also can be considered to include correlations and other results of analyses of the data sets. Thus, a pattern can include a plurality of different elements as described above, or can include vector quantities derived from the elements.

A “biological state” refers to a condition in which a biological system exists, either naturally or after a perturbation. Examples of a biological state include, but are not limited to, a normal or healthy state, a disease state, including both physical and mental disease, a stage of disease progression or resolution, a pharmacological agent response (e.g., drugged and healthy or drugged and diseased), various different toxic states, a biochemical regulatory state (e.g., apoptosis), an age response, an environmental response, and a stress response. The biological system preferably is mammalian, which includes humans and non-human mammals such as mice, rodents, guinea pigs, dogs, cats, monkeys, and the like.

A pattern of a biological state permits the comparison of patterns to determine whether the animals from which the samples and patterns were derived are in the same or different states, e.g., a healthy or a diseased state. A biological system is often better characterized using a multivariate analysis rather than using multiple measurements of the same variable because multivariate analysis envisions the biological system in greater detail, and takes into account biology at the systems level. Disparate data from multiple, different sources is treated as if in a single dimension rather than in multiple dimensions. Consequently, the analysis of data as disclosed herein is more informative and typically provides a pattern that is more robust and predictive than one that is developed by systematically evaluating multiple components individually or relies on one particular type of biomolecule.

The data sets used in the pattern or methods of the invention may include data obtained from measurements that do not detect concentrations of biomolecules, either in addition to or in place of such concentration data. For example, data from psychiatric evaluations, electrocardiography, computed axial tomography, positron emission tomography, x-ray, and sonography may be employed in data sets herein.

In various embodiments of the invention, data sets employed in the methods or patterns described herein include data on at least 10, 100, 1000, 10,000, or even 100,000 biomolecules, all of which may be represented as individual elements or cells in a pattern.

A “type of biomolecule” refers to a class of biomolecules generally associated with a level of a biological system. For example, genes and gene transcripts (which may be interchangeably referred to herein) are examples of types of biomolecule that generally are associated with gene expression in a biological system, and where the “level” of the biological system is referred to as genomics or functional genomics. Proteins and their constituent peptides (which may be interchangeably referred to herein), are another example of a type of biomolecule that generally is associated with protein expression and modification, and where the “level” of the biological system is referred to as proteomics. Another example of a type of biomolecule is metabolites (which also may be referred to as small molecules), which generally are associated with a level of a biological system referred to as metabolomics.

A “biological sample type” includes, but is not limited to, blood, blood plasma, blood serum, cerebrospinal fluid, bile acid, saliva, synovial fluid, pleural fluid, pericardial fluid, peritoneal fluid, sweat, feces, nasal fluid, ocular fluid, intracellular fluid, intercellular fluid, lymph, urine, and cell or tissue extracts from, for example epithelial cells, endothelial cells, kidney cells, prostate cells, blood cells, lung cells, brain cells, adipose cells, tumor cells, and mammary cells. The sources of biological sample types may be different subjects; the same subject at different times; the same subject in different states, e.g., prior to drug treatment and after drug treatment; different sexes; different species, e.g., a human and a non-human mammal; and various other permutations. Further, a biological sample type may be treated differently prior to evaluation such as using different work-up protocols.

Measurement techniques for acquisition of data include, but are not limited to, mass spectrometry (“MS”), nuclear magnetic resonance spectroscopy (“NMR”), liquid chromatography (“LC”), gas chromatography (“GC”), high performance liquid chromatography (“HPLC”), capillary electrophoresis (“CE”), gel electrophoresis (“GE”) and any known form of hyphenated mass spectrometry in low or high resolution mode, such as LC/MS, GC/MS, HPLC/MS, CE/MS, MS/MS, MSⁿ, and other variants. Measurement techniques include biological imaging such as magnetic resonance imagery (“MRI”), video signals, and an array of fluorescence, e.g., light intensity and/or color from points in space, and other high throughput or highly parallel data collection techniques. Measurements may also be taken via various assays including parallel hybridization assay, parallel sandwich assay, and competitive assay.

Measurement techniques also include optical spectroscopy, digital imagery, oligonucleotide array hybridization, protein array hybridization, DNA hybridization arrays (“gene chips”), immunohistochemical analysis, polymerase chain reaction, nucleic acid hybridization, electrocardiography, computed axial tomography, positron emission tomography, and subjective analyses such as found in text-based clinical data reports. For a particular analysis, different measurement techniques may include different instrument configurations or settings relating to the same measurement technique.

A “data set” includes measurements derived from one or more sources. For example, a data set derived from a measurement technique includes a series of measurements collected by the same technique, i.e., a collection or set of data of related measurements. Further, data sets may represent collections of diverse data, e.g., protein expression data, gene expression data, metabolite concentration data, magnetic resonance imaging data, electrocardiogram data, genotype data, single nucleotide polymorphism data, and other biological data. That is, any measurable or quantifiable aspect of a biological system being studied may serve as the basis for generating a given data set.

A “feature” of a data set refers to a particular measurement associated with that data set that may be compared to another data set. For example, a pattern typically is a set of data features that permit characterization of a biological state.

Data sets may refer to substantially all or a sub-set of the data associated with one or more measurement techniques. For example, the data associated with the spectrometric measurements of different sample sources may be grouped into different data sets. As a result, a first data set may refer to experimental group sample measurements and a second data set may refer to control group sample measurements. In addition, data sets may refer to data grouped based on any other classification considered relevant. For example, data associated with the spectrometric measurements of a single sample source may be grouped into different data sets based on the instrument used to perform the measurement, the time a sample was taken, the appearance of a sample, or other identifiable variables and characteristics.

In addition, it should be realized that the term “data set” includes both raw spectrometric data and data that has been preprocessed, e.g., to remove noise, to correct a baseline, to smooth the data, to detect peaks, and/or to normalize the data.

“Statistical analysis” includes parametric analysis, non-parametric analysis, univariate analysis, multivariate analysis, linear analysis, non-linear analysis, and other statistical methods known to those skilled in the art. Multivariate analysis, which determines patterns in apparently chaotic data, includes, but is not limited to, principal component analysis (“PCA”), discriminant analysis (“DA”), PCA-DA, canonical correlation (“CC”), cluster analysis, self organizing mapping (“SOM”), partial least squares (“PLS”), predictive linear discriminant analysis (“PLDA”), neural networks, and pattern recognition techniques.

Other features and advantages of the invention will be apparent from the following description and the claims.

### BRIEF DESCRIPTION OF THE DRAWINGS

FIGS. 1A-1D are MSIs produced from data obtained from LC/MS analysis of mammalian samples. FIG. 1A shows MSIs from healthy mammals that had been administered vehicle; FIG. 1B shows MSIs from healthy mammals that had been administered a drug; FIG. 1C shows MSIs from diseased mammals that had been administered vehicle; and FIG. 1D shows MSIs from diseased mammals that had been administered the drug. Distinctions among these groups are readily observed based on MSI differences.

FIG. 2 is a molecular pathology map for an atherosclerosis disease model. ApoE3-Leiden transgenic mice were used as an animal model of atherosclerosis as described in Example 12. The molecular pathology map separates the transgenic mice (labeled TG#) from the wild type mice (labeled WT#) in an unsupervised manner.

FIG. 3 is a table of disease pathology scores for 19 animals used in a study of atherosclerosis (Example 12).

FIG. 4 is a set of 19 molecular systems images (MSIs), for animals used in a study of atherosclerosis (Example 12). The numbers in parentheses (s=##) are the atherosclerosis pathology scores of each animal.

### DETAILED DESCRIPTION OF THE INVENTION

The methods described herein rely on measurements of biological samples, including analysis of metabolites, proteins, and/or genes and gene transcripts, for the production of patterns of biochemical activity or subjects in a population. Understanding a biological system, either as a whole or a subset thereof, can improve multiple aspects of pharmaceutical discovery and development, including drug safety and efficacy, drug response, the etiology of disease, and diagnosis and treatment of disease. A systems biology platform can integrate genomics, proteomics, and metabolomics, and bioinformatics, and results in a data integration and knowledge management platform that generates connections, correlations, and relationships among thousands of measurable biomolecules to develop a pattern of a biological state. Resulting patterns can be combined with clinical information to increase the knowledge of a biological state.

The methods described herein may be used to develop a pattern of a biological state based on one or more types of biomolecules. Patterns of types of biomolecules facilitate the development of comprehensive patterns of different levels of a biological system, and permit their integration and analysis. The methods may be used to analyze measurements derived from one or more biological sample types, one or more measurement techniques, one or more types of biomolecules or a combination thereof to permit the evaluation of similarities, differences, and/or correlations in biological states. From these measurements, better insight into underlying biological mechanisms may be gained, novel biomarkers/surrogate markers may be detected, and intervention routes may be developed.

The methods described herein involve the production of patterns based on differences and similarities in the concentrations of biomolecules across a plurality of data sets. Thus, an aid to the practice of the invention is the availability of data from a study set that includes a group of individuals selected so as to isolate to the extent possible the differences between the biological state under study from controls, and to eliminate from consideration biochemical changes involved in all other biological states. Conditions are typically set so as to isolate the variable under study. Thus, members of the study set can be segmented into two or more groups based on the phenotypic differences under study but otherwise be phenotypically similar. To the extent the members of the study set differ in aspects of their biological state separate from the state under study, the results may deteriorate, and noise may mask signal.

Furthermore, the raw data used to produce these patterns may be, and typically are, preprocessed to assist in the comparison of different data sets. In particular, to compare data across different types of biomolecules, appropriate preprocessing can be performed. Preprocessing of the data may include (i) aligning data points between data sets, e.g., using partial linear fit techniques to align peaks of spectra of different samples; (ii) normalizing the data across the data sets, e.g., using standards in each measurement to adjust peak height; (iii) reducing the noise and/or detecting peaks, e.g., setting a threshold level for peaks so as to discern the actual presence of a species from potential baseline noise; and/or (iv) other data processing techniques known in the art. Data preprocessing can include entropy-based peak detection as disclosed in U.S. Pat. No. 6,743,364, and partial linear fit techniques (such as found in J. T. W. E. Vogels et al., “Partial Linear Fit: A New NMR Spectroscopy Processing Tool for Pattern Recognition Applications,” Journal of Chemometrics, vol. 10, pp. 425-38 (1996)).

The methods described herein generally include evaluating with statistical analysis a plurality of data sets and comparing features among the data sets to determine one or more sets of differences to develop a representation of a biological state based on the comparison. Of course, not all data in such a dataset will be relevant to the biological system under investigation. Accordingly, to improve the resolution of a pattern, e.g., an MSI, it is helpful to filter the data using methods known per se to remove data indicative of biomolecule concentration that is static across all subjects, random, or otherwise does not change as between test subjects and controls in a way that is relevant to the biochemistry of the biological state under study. This can be done using methods such as univariate and multivariate statistics, parametric statistics, non-parametric statistics to e.g. discern data features which do not change in a statistically significant manner, and queries of public or private databases or scientific literature to assess the relevance of a measured biomolecule to the biological state under study. In some embodiments, the data sets are derived from one or more biological sample types and include measurements derived from one or more measurement techniques. In other embodiments, the data sets are derived from two or more biological sample types and include one or more different types of spectrometric measurements of a sample of the biological system.

Measurements for a particular type of biomolecule usually are generated by a measurement technique or techniques that are often used and known in the art for that particular type of biomolecule. For example, an analysis of metabolites may use NMR, e.g., ¹H-NMR; LC/MS; GC/MS; and MS/MS. Analysis of other types of biomolecules may use LC/MS; GC/MS; and MS/MS.

In one embodiment, the method involves selecting a biological sample; preparing the biological sample based on the biomolecules to be investigated and the measurement techniques to be employed; measuring the biomolecules in the biological sample; optionally preprocessing the raw data; placing individual data points in a virtual or real position so as to produce a pattern or image using a previously determined mapping key or table embodied in software; and then analyzing the pattern or image to identify the biological state of the subject from whom the sample was taken. The methods may also include normalizing a plurality of data sets or averaging a plurality of data sets to facilitate comparison of the data across types of biomolecules and across biomolecules whose concentrations vary over different ranges. The mapping key directing placement of the data points is derived from a study set, and often the analysis includes comparing the subject generated pattern or image to a pattern or image made from the data used to produce the study set or from multiple samples taken from subjects in known biological states. The use of a plurality of data sets as a study set to determine a suitable mapping key or table is described below, and may be adapted from the literature of data mining and processing techniques.

Normalization model. A method for normalizing biomolecule concentration data, such as expression data, protein data, and metabolite level data is now described. A sample variety effect, an array effect, and a dye effect are introduced into a log-linear model, and a maximum likelihood maximization technique is applied to calculate all the parameters of the model and determine the optimal scaling factor for each array and dye. The normalization method is generic and can be applied to a variety of data, experimental setups, and designs. The model described below uses terminology from gene expression analysis. For example, the “array” in proteomics experiment could be one mass spectrometer run, and the “dye” could describe all samples used during the single run. Nevertheless, other types of biomolecules could be analyzed using the model described below.

The data matrix x is characterized by the gene index g(g=1 . . . . N_{g}), array index i(i=1 . . . Nᵢ), dye index k(k=1 . . . . Nₖ), and the variety index v(v=1 . . . Nᵥ). For each variety v, there are Cᵥ samples corresponding to it, so Nₛₐₘₚₗₑₛ=σᵥ Cᵥ=NᵢNₖ. Since variety assignment is a function of array and dye indices, each data point is uniquely described by indices g, i, and k. For convenience the matrix is transformed logarithmically:
y_{gik}=log(x_{gik}). (1)
Data is described by the following model:
y_{gik}=μ_{gv}+Aᵢ+Dₖ+ε_{gik}, (2)
where the gene and variety effects are described by μ_{gv}, the array effect by Aᵢ, the dye effect by Dₖ, and the error function by ε_{gik}. The error function is assumed to be normally distributed with zero mean and the variance σ_{gv}², i.e., the variance is permitted to be different for each gene and variety. The variety index v is a unique function of i and k, and can be written as {i,k}εv. Since the gene and variety, array, and dye effects are assumed to be fixed, the distribution of expression levels can be described as:

$$P ( {y}_{gik} \vert {μ}_{gv} , {A}_{i} , {D}_{k} , {σ}_{gv}^{2} ) = \frac{1}{\sqrt{2 π {σ}_{gv}^{2}}} exp ( - \frac{{( {y}_{gik} - {μ}_{gv} - {A}_{i} - {D}_{k} )}^{2}}{2 {σ}_{gv}^{2}} ) . \qquad ( 3 )$$

A maximum likelihood estimation is used to calculate the optimal scaling parameters used to properly normalize the data. Solving for the parameters μ_{gv}, Aᵢ, Dₖ, and σ_{gv} leads to the following equations:

$${{u}^{⋒}}_{gv} = \frac{1}{{C}_{v}} {\sum }_{ik ∈ v} ( {y}_{gik} - {{A}^{⋒}}_{i} - {{D}^{⋒}}_{k} ) , \\ {{A}^{⋒}}_{i} = \frac{1}{{N}_{i}} {\sum }_{gk} ( {y}_{gik} - {{μ}^{⋒}}_{gv} - {{D}^{⋒}}_{k} ) , \\ {{D}^{⋒}}_{k} = \frac{1}{{N}_{k}} {\sum }_{gi} ( {y}_{gik} - {{μ}^{⋒}}_{gv} - {{A}^{⋒}}_{i} ) , \\ {{σ}^{⋒}}^{2} = \frac{1}{{N}_{g} {N}_{i} {N}_{k}} {\sum }_{ik ∈ v} {( {y}_{gik} - {{μ}^{⋒}}_{gv} - {{A}^{⋒}}_{i} - {{D}^{⋒}}_{k} )}^{2} . \qquad ( 4 )$$

The optimal scaling factors for each array and dye are then:
sᵢₖ=−Aᵢ−Dₖ, (5)
so the normalized expression levels are:
\overline{x}_{gik}=x_{gjk}× exp(sᵢₖ) (6)

Significance tests and bootstrap methods. The normalized data may be compared to a null model, and a p-value may be calculated that measures the probability that the deviation of the data from the null model can be attributed to the random error. The parameter used for comparison is the fold ratio between the two chosen varieties. To evaluate the method, a t-test is performed to compare the two chosen varieties. [Sheskin, Handbook of Parametric and Nonparametric Procedures, Chapman & Hall/CRC, Boca Raton, Fla. (2000).] The corresponding p-values can be calculated for each biomolecule. When assessing the statistical significance of fold change for each biomolecule, one needs to take into consideration the total N_{g} p-values calculated, as several p-values with p<1/N_{g} are expected. To account for this, the overall likelihood, P(p), of observing a p-value≦p for any of the N_{g} biomolecules is used. Assuming independence of all biomolecules, the overall likelihood is estimated with:
P(p)≈1−(1−p)^{Ng}. (7)

Assuming independence of biomolecules is an oversimplification, and a more accurate way to calculate p-values and P(p) values is by using the bootstrap method with the parameters. (μgv,Aᵢ, Dₖ, σ_{gv}) of the null model being used to general random data sets.

This and other standard methods for significance testing can be used to determine whether a particular variable should be included in a pattern, e.g., an MSI. This can be important to eliminate variables that are not indicative of any state of interest to the practitioner. For example, it is possible for a measured variable to be totally random, and therefore not provide any information about the sample at all. Such variables will be eliminated by significance testing methods such as the above.

Significance testing can also be used to ease interpretation of patterns, e.g., MSIs, by presenting only a subset of the effects that occur on a particular pattern. For example, in systems pathology, it may be desirable to focus only on the difference between a particular diseased and normal state. In this case, only variables found to significantly discriminate between these two states may be included in the pattern. Similarly, in some cases of systems pharmacology, it may be desirable to display the effect of a drug on only those variables that discriminate between disease and normal, and thus highlight effects of the drug on the disease, while eliminating effects of the drug on non-disease variables.

Clustering

Data sets including values indicative of the concentration of biomolecules in one or more organisms may be organized by an unsupervised clustering algorithm, e.g., a Self Organizing Map (SOM) algorithm, a Sammon plot algorithm, or an elastic net algorithm. Preferably, the clustering produces a pattern such as a multidimensional image, e.g., a two-dimensional grid, in which the location of elements, e.g., pixels, relative to one another, is indicative of the degree of correlation between the data represented by the element for a given biological state or within a group of organisms. Alternately, the location of the elements of the multidimensional image may be indicative of the degree of second moment, third moment, or higher moment correlations or partial correlations between the data.

Unsupervised clustering requires multiple data sets for use in training the program. These data sets can be generated using known techniques for analyzing multiple analytes, from one or more samples, from multiple organisms or multiple samples from the same organism at different time points. The identity of the biomolecules being analyzed is not critical, except that at least some of them must be indirectly or directly involved with the biochemistry underlying the biological state of the organism being analyzed. Knowledge of the identity of the biomolecules is not required, although such information may be useful, as described herein. Preferably, at least some and preferably half of the animals/humans involved in the study exhibit symptoms/phenotype/characteristics relevant to the biological state under study.

As an illustrative protocol, data is obtained from 16 rodents, eight of which are diseased, and eight of which are healthy. Blood or urine samples are taken from each rodent and analyzed by, for example, LC/MS. After filtering the data, the relative concentration of 576 detectable molecular species is then determined using standard means. Each rodent then is administered a drug known to treat the disease, and the sampling, analyses, and filtering is repeated. In certain instances, a single biomolecule may be represented by multiple peaks in a LC/MS analysis depending on the fragmentation of the biomolecule, and thus two or more species detected in a LC/MS may represent a single biomolecule. For the purposes of this example, we assume no such redundancy in the data; in an actual analysis, such redundancy may be used to increase the internal consistency of the clustering. This analysis produces a dataset that can be arranged in a table having 32 columns, each column containing data from one rodent (eight diseased—no drug, eight diseased—drugged, eight healthy—no drug, and eight healthy—drugged) and 576 rows, each row representing a particular biomolecule. The order of placement of the biomolecules in the table or the order of placement of the rodent individuals under study is immaterial, as long as they are consistent (e.g., each row contains data on the same biomolecule for each rodent sample, and all the data in a column is from the same rodent sample).

The data are normalized by assigning the lowest value of a biomolecule in a row −1 and the highest value +1, (or other arbitrary units) with intermediate values assigned to values in between. Alternatively, one can normalize by looking only at the normal healthy rodent data, determine an average value for each biomolecule, and define that value as zero for that biomolecule, then devise a scale from −10 to +10, and rank all other data in that row on the scale. In other embodiments, a logarithm or other function of the data may be taken. Software programs are available for automated normalization based on the desired method.

These normalized data are now used to produce a study set of 576 “plots” for use in an unsupervised clustering program. These plots can be described as a graph plotting the normalized value for a biomolecule detected by LC/MS as a function of each of the thirty-two rodent samples. A given plot might have rodent number (1 through 32) on its abscissa and level of biomolecule on its ordinate. These plots are then assessed for similarity, e.g., by calculating the correlation coefficient for each plot or by summing the square of the differences. An algorithm (such as an SOM program) then is applied to arrange each plot into an element (cell or pixel) of a pattern. The algorithm virtually shifts the location of each plot on the grid to search for an arrangement wherein plots in adjacent pixels are as similar to each other as possible. Rather than each element being placed at random, it is placed such that its neighbors have values similar to it, and there are preferably no sharp discontinuities in the pattern. Different algorithms may produce different solutions, and the same algorithm on occasion (depending on its logic) may produce different solutions.

Each of the 576 biomolecules detected has now been assigned to a pixel or cell in a two (or more) dimensional space based on the similarity of change of normalized concentration of each biomolecule across the samples, and a table or mapping key has been produced assigning each biomolecule to a specified location. The data set now can be visualized as a pattern, e.g., as a table listing the biomolecule and its position, e.g., its x and y coordinate, or as a plot which can be visually or computationally inspected. The derived mapping key or table now may be used to assign the position of each data point representative of biomolecules from a sample from any individual subject in the study set, or a new test animal and to produce patterns which can yield information concerning the biological state of the animal. Thus, the mapping key can now be used to assign normalized data points from any rodent sample that measures the same biomolecules, or another sample that measures the same or homologous biomolecules, to a particular coordinate in the pattern. Thus, once the location of the biomolecules in the pattern is determined, a molecular systems image (MSI) for an organism in a given biological state can be produced. Data from the 576 biomolecules of any rodent, or potentially an organism having the same or homologous biomolecules, may now be imaged according to the mapping key produced by the study set. This pattern can be recognized as characteristic of the biological state of that rodent, or other organism. The pattern can also be presented so as to be visually observable by assigning color or other indicia related to the relative concentration measured for each biomolecule.

A molecular pathology map may be produced using the same or a similar process, except that each pixel or cell in the image represents a different sample, e.g., each from a different animal, instead of a different biomolecule, and the key or table is produced from the study set by applying a clustering algorithm to normalized profiles of biomolecule concentration within each sample. Such a pattern may reveal clusters of animals, e.g., reveal distinctions among animals exhibiting a similar phenotype based on different biochemical profiles.

Methods

It has now been discovered that patterns produced as disclosed herein, particularly such patterns generated from data derived from different types of samples from a given organism, data obtained from different analysis techniques, data indicative of the concentrations of different types of biomolecules sampled from a given organism, and particularly data sets derived from various combinations of such diverse assessments of an organism's biochemistry, are indicative of the biological state of the organism and can reflect differences too subtle to be observed otherwise. Such patterns have a variety of uses, e.g., in drug discovery, drug development, medical diagnosis, medical treatment, and toxicology. In one embodiment, a pattern obtained from an organism, e.g., a human, is compared to another pattern obtained from an organism, which may be the same organism, a different organism of the same species, or an organism of a different species. Alternatively, a pattern from an organism may be compared to a composite pattern, e.g., produced from the average or other combination of data from multiple organisms. Patterns may be compared by computer or by visual analysis, e.g., in the form of two-dimensional images produced by the methods disclosed herein. The elements that make up a pattern, e.g., the pixels in an image, may also be linked to information on the data, e.g., biomolecules, represented, e.g., the identity if known, or information on the raw data concerning the biomolecule. The identity of unknown biomolecules that are located in particular elements of a pattern that are indicative of a biological state may also be determined, if desired. For example, if a particular region of a pattern is determined to be indicative or characteristic of the biochemistry which results from a disease or adverse effect of treatment, the identity of the biomolecules in that region may be determined by further qualitative analysis of the samples to understand the biochemical mechanisms involved.

A pattern also may be combined with a numerical score. A number can serve to place the dataset from a given individual on a line of arbitrary length, expressed as a number, and displayed together with the pattern. Samples in the same biological state have numbers in the same region on the line. The number may be determined using any one of a number of known data analysis techniques such as linear or non linear classification or clustering metrics. These data analysis techniques are well known and are often embodied in data analysis software which determine Euclidean distance, correlation distance (Pearson Correlation or rank correlation), Manhattan distance, weighted harmonic distance, Chebychev distance, or principal component score distance.

Many of the novel uses of patterns described herein involve the development of a reference pattern, e.g., an image, and then comparing that reference pattern to a pattern obtained from an organism, where the data in both patterns are arranged in the same order. Such a comparison allows for the determination of differences or similarities between the reference pattern and the pattern obtained from the organism. The following discussion provides exemplary uses for these comparisons.

Pharmacology. Patterns or images produced from clustered data (including molecular systems images, their underlying data precursors, and groups of biological markers) are useful for studying the effects of a drug, combinations of drugs, and drug candidates on the biological state of an organism. A drug, drug candidate, or combination of drugs or drug candidates can be administered to a healthy or diseased organism, and a pattern showing the relative concentration of biomolecules from the healthy or disease organism can be compared to a reference, e.g., an unmedicated healthy or diseased organism or an organism medicated at a different dosage, manner, or time. For example, a drug or combination of drugs can be administered to a diseased organism, and an MSI is produced from the treated organism and compared to a reference MSI representing a healthy organism or one from a diseased organism treated successfully with a known drug. The efficacy of the drug can then be determined from the degree of similarity between the two patterns. Such determinations of efficacy can also be used to identify second medical uses of existing drugs and combinations of drugs, e.g., known drugs, that show a synergistic therapeutic effect or a previously unknown therapeutic effect. Patterns of the effects of drugs or drug candidates on a diseased and healthy organism, e.g., in a library, can also be used rationally to select effective drugs or combinations of drugs that would produce a profile similar to a healthy or effectively drugged diseased organism if administered to a diseased organism. In addition, patterns produced from the administration of drug candidates or drugs not known to be effective against a disease may be compared to a pattern produced by administration of a drug with a known efficacy against that disease. Comparison of patterns may also be used to evaluate drugs or rank drug candidates based on toxicity, potency (dosage), bioavailability, duration of action, and the frequency or severity of a side effect when compared to an appropriate reference, sometimes more conveniently and easily than multiple animal experiments and observations of results. For example, patterns produced from the administration of multiple doses of a drug may be employed to assess the dose response of an organism and assess therapeutic index (dose range between minimally efficacy and unacceptable toxicity). Patterns may also be used to develop surrogate end points (a “success profile”) useful to evaluate drug molecule candidates or effects in individuals in clinical trials.

Patterns, e.g., MSIs, may also be employed to permit better assessment of a drug candidate's efficacy and toxicity in humans based on animal studies. For example, profiles can be correlated between clinical trial participants who have a particular outcome and animals exhibiting the same outcome, and one could administer a drug that is successful in humans to an animal and develop an MSI of its effect in the animal. In this circumstance, a drug candidate that, when administered to an animal, replicated the MSI produced from the known drug would be suggestive of efficacy in humans.

Furthermore, the use of MSIs provides a way to determine whether individual drugs in a collection of candidates under development for a single disease, all of which have been shown to be active in standardized assays, operate through the same or differing mechanisms of action, so as to avoid costly unwitting duplication of effort. The use of MSIs also allows for discovering a superior drug with an unknown target or mode of action (e.g., by determining which molecules can replicate a successful end point profile).

Toxicology. Patterns may also be used to determine whether a drug, drug candidate, or combination of drugs cause toxicity, e.g., liver, kidney, or nerve toxicity. For example, a pattern such as an MSI obtained from an organism which has received a dose of the candidate drug preparation can be compared to an MSI generated from a reference sample from the same or a different individual organism known to have exhibited a particular toxicity, e.g., having been administered a drug with a known toxic effect. Measures of toxicity allow for the selection of drugs with reduced toxicity compared to other potential therapies, or for the addition of other therapeutic agents that reduce the toxicity for a drug that is active against a particular disease. In addition, the evaluation of toxicity may be used to reveal whether a molecule's toxicity is inexorably linked to its efficacy (in which case it and perhaps its target may be abandoned).

Diagnostics. Patterns generated from diseased organisms may be indicative of the disease state and can be used, e.g., to examine a patient for the presence of, stage of, severity of, diagnosis of, therapy options for treatment of, or prognosis for a pathological phenotype. For example, an MSI produced from a sample from an individual presenting phenotypic signs of disease or morbidity can be compared for diagnostic purposes to reference MSIs previously generated and known to be characteristic of the disease, its state of progression, a subtype of the disease, or MSIs from plural diseases that produce the same or a similar phenotype. Such a diagnosis is useful in choosing among therapeutic courses.

Patterns can also be used to segment phenotypically similar diseases into subspecies of the disease which are biochemically distinct, and which are best addressed by different treatment options or drugs. Elements of such patterns represent data from individual organisms exhibiting the phenotypic symptoms. Distinct clusters of individuals within the map are indicative of different subspecies of disease, e.g., based on a different biomolecular basis that produce similar phenotypes.

### EXAMPLE 1

### Identification of Therapeutic Efficacy

In this example, the study set comprises individuals who are confirmed as suffering from a given disease and healthy individuals. A pattern having elements representative of the concentrations of biomolecules in samples drawn from the patients then is produced by an SOM or other suitable clustering software, and a mapping key is developed. The mapping key is applied to data from individual healthy patients or to composite data from a plurality of healthy subjects to produce a “health” or normal pattern. Similarly, the mapping key is applied to the data from confirmed diseased subjects or to composite data from a plurality of diseased subjects to produce a “diseased” pattern. A drug candidate, drug, or combination of drugs then is administered to a diseased, phenotype matched patient. One or more samples taken from the patient are analyzed to produce data which is filtered, normalized, and treated with the mapping key to produce a pattern, in the same way the study set was treated. This pattern then may be compared with the healthy and diseased reference patterns. A similarity between the “healthy” reference pattern and the pattern from the patient is indicative of therapeutic efficacy of the drug, drug candidate, or drug combination against the disease. Patterns characteristic of the effects of a drug on a healthy patient, and of a diseased patient successfully treated with a drug may also be used to determine therapeutic efficacy. Such patterns when used as references can help to determine whether the drug under test affects in a healthy individual the same biomolecule concentrations that are abnormal in the diseased individual. This method also can be used for repurposing drugs by determining if a drug known for treating one disease may be used to treat other diseases. Another use of the method is to determine if combinations of drugs have efficacy, perhaps where neither alone would be efficacious.

### EXAMPLE 2

### Use of Perturbagens

Because the methods of the invention allow assessment of the biochemical effects of compounds, a small dose of a compound, a “perturbagen,” can be administered to probe the biochemical nature of the disease or to determine if that compound affects the biochemistry of a subject in a desirable or undesirable way. This aspect of the invention may be used productively to diagnose and find an effective therapeutic regimen to treat mental disease such as depression, bipolar disorder, or schizophrenia. A perturbagen typically is a sub-therapeutic and sub-toxic dose of a compound, which can either be a drug or a surrogate for a drug, e.g., a compound known to be metabolized like the drug in question administered in a sub-toxic dose. Perturbagens may be administered to humans in appropriate circumstances and to laboratory animals.

This method allows for the probing of efficacy or toxicity with minimal safety concerns. One or more subjects are administered a perturbagen, and data on the concentration of biomolecules are then obtained from a relevant sample taken from the subject. After filtering and normalizing, a mapping key developed by a clustering algorithm on an appropriate study set is applied to the data to produce a pattern, which optionally is converted to a visually observable image. The image created is indicative of the effect of the perturbagen on the subject, as judged by comparisons with MSIs generated from subjects in the study set having known biological states. This in turn may be suggestive of a particular diagnosis, suggestive that a particular drug is likely to be most effective in treating the disease, or suggestive that a particular drug should be avoided. Furthermore, new compounds that affect the biomolecules in the subject in a manner consistent with a therapeutic efficacy can then be further tested, and compounds that affect the biomolecules in a subject in a manner consistent with toxicity or no therapeutic effect can be discarded.

### EXAMPLE 3

### Determination of Dose Response

A drug is administered in a several dosages to multiple subjects. Data on the concentration of biomolecules are then obtained from the subjects and from controls. An SOM algorithm is used to create a pattern of biomolecules (a mapping key) from a plurality of data sets to determine the order of elements in the pattern, where each element represents one or more biomolecules. The data from individual drugged subjects are then ordered according to the mapping key or table created by the SOM algorithm. The pattern created may be compared with the pattern of healthy subjects or successfully drugged subjects and is indicative of the effect of a particular dosage on a subject. For example, it may be that a pattern indicative of a healthy state is achieved at one dose, but smaller doses cannot achieve this biological state, and larger doses rapidly become toxic. By studying a variety of dosages systematically, appropriate dosage levels balancing therapeutic efficacy and minimal toxicity can be determined. The method may also be used to study if a particular dosage causes toxicity. In addition, this method may be used to determine the therapeutic index of a drug.

### EXAMPLE 4

### Molecular Effects of Drugs

A reference MSI is produced indicative of successful drug therapy of a subject, where the type of drug administered has a known effect, but an unknown mechanism. Now candidate compounds can be administered to subjects, data acquired from samples, and MSIs generated using a protocol parallel to that used to create the reference MSI. These can be compared to the reference MSI to determine the effects of the candidate compounds. A similarity between the pattern produced by the candidate drug and the reference is indicative of a similarity in biological response and therefore suggestive of efficacy or of a common mechanism of action. In addition, when the pattern produced by the drug is compared to a reference pattern, individual biomolecules that show differences or similarities in concentration can be identified and examined to provide further insight into the mechanism of action.

### EXAMPLE 5

### Identifying Responders and Non Responders

A group of patients that have been administered the same drug or combination of drugs is studied. Data on the concentration of biomolecules are obtained from each patient in the population and from controls receiving no drug. An SOM algorithm then is applied to the data to create a pattern, in which the individual elements represent one or more patients, as opposed to biomolecules. Distinct clusters of patients are observable in the pattern for every different type of effect of the drug on the subjects. For example, a single drug, or combination, may provide a therapeutic effect in one subpopulation of patients but be toxic or ineffective in another population. Once the subjects are clustered, data from representative subjects, or average data from the subjects in a single cluster, may be used to develop molecular systems images in which the elements of a pattern represent biomolecules, thereby providing a pattern that is indicative of the particular effect of a drug, e.g., a positive response, in that type of subject. Such studies are of use in clinical trials and prior to the administration of a drug or drugs. In clinical trials, if adverse effects are observed in a subset of patients, the methods described can be used to determine which patients likely will respond negatively before drug administration after administration of a perturbagen. This permits one to segregate the population to exclude non responders from the study. Similarly, if a drug is known to cause adverse events in some patients, the patients can be screened prior to the administration of the drug or after administration of a perturbagen to determine whether they are candidates for administration of the drug or toxic responders. In addition, with some drugs, it becomes apparent only after an extended period of use of the drug that certain adverse events will occur, or that the patient will benefit. Thus, a patient may be determined to be a responder or a non responder as indicated by a characteristic MSI, generated with or without a perturbagen, before administration of any drug, or may be monitored by generation of MSIs periodically during the course of treatment to determine whether drug treatment should be continued.

### EXAMPLE 6

### Development of Surrogate Markers

Subjects having a known biological state are studied, e.g., the subjects have been diagnosed with a known disease or toxicity, or have been administered a known drug to achieve an effect. Data on the concentration of biomolecules are obtained from the subjects and from control subjects. After filtering and normalizing the data an SOM algorithm is used to create a pattern of biomolecule concentrations from the data sets to determine the order of biomolecule elements in a pattern so as to produce a mapping key. Data from a subject known to be in the biological state under study are then ordered according to the same mapping key to produce a pattern generated by assigning the position of each data point in accordance with the mapping key as determined by the SOM algorithm applied to the teaching set. The pattern created from the subject can be used as a surrogate marker which, if found in a patient, indicates that the patient is in the biological state. Stated differently, the pattern produced is indicative of the biochemical characteristics of the biological state in that individual. Data from a population of subjects in the same state may also be averaged or otherwise combined to produce a composite pattern. A sample from a subject in an unknown biological state can then be analyzed in a way parallel to the analysis and data treatment used in development of the study set. When the mapping key is applied to the data, an MSI is produced and then compared to one or more surrogate marker MSIs to determine whether the subject is in a particular biological state. Such comparisons are useful for determining health, disease, toxicity, or the effects of drugs.

In another example, a known drug with a known effect in humans is administered to non-human experimental animals such as rats to develop a pattern or MSI which acts as a surrogate marker for the effect of that drug in rat. This surrogate marker can be used in comparisons with patterns or MSIs produced in rats after administration of drug candidate compounds, e.g., to determine whether a candidate compound can produce a similar MSI or pattern, and therefore potentially may have a therapeutic effect in humans similar to that of the known drug.

### EXAMPLE 7

### Diagnosis of Disease

A pattern having elements representative of the concentrations of biomolecules prepared as set forth herein from relevant samples from confirmed diseased individuals may be used as a diagnostic pattern, e.g., as a diagnostic reference MSI. Several different diagnostic reference patterns may be prepared, all of which are indicative of the biochemistry of the disease, but which differ in other phenotypic traits. For example, there may be different MSIs for the same disease in males, females, immune compromised individuals, obese individuals, etc. Then, a patient presenting with disease symptoms, or otherwise suspected of having a disease or propensity for a disease, can be diagnosed by collecting a relevant sample, such as serum, which is analyzed to produce data on the concentration of biomolecules therein. The data are filtered, normalized, and assigned positions in a field or volume to generate a pattern. This can be compared with one or many reference patterns to produce valuable diagnostic insight. A similarity between the pattern of the subject and a reference pattern is then indicative of a potential diagnosis.

### EXAMPLE 8

### Methods of Identifying Sub-Types of Diseases

Subjects that exhibit the same or similar disease symptoms are studied. Data on the concentration of biomolecules are obtained from each subject in the population. After filtering and normalizing the data, an SOM algorithm is applied to create a pattern, in which the individual elements represent one or more subjects, as opposed to biomolecules. Distinct clusters of subjects are observable in the pattern for every biochemically distinct disease that produces the same symptoms. Such patterns may be used to identify sub-types of diseases, and thereby, focus treatment on the underlying cause. Once the subjects are clustered, data from representative subjects, or average data from the subjects in a single cluster, may be used to develop molecular systems images in which the elements of a pattern represent biomolecules, thereby providing a pattern that is indicative of the biochemical effect of each distinct disease on a subject.

### EXAMPLE 9

### Comparison of Molecular Mechanisms of Drugs

A plurality of drugs, or drug candidates, that treat the same disease is administered to a population. Data on the concentration of biomolecules are obtained from controls and from each subject in the population, where each subject has been administered one drug (or combination of drugs as a single therapeutic intervention). An SOM algorithm is then applied to the data to create a pattern, in which the individual elements represent one or more subjects, as opposed to biomolecules. A distinct cluster of subjects is observable in the pattern for each drug that acts through the same biochemical mechanism. For instance, if five drugs are given, and each drug acts on an independent biochemical pathway to produce a therapeutic effect, then five distinct clusters will be observable in the pattern. If five drugs are given, and each drug acts on the same pathway, then only one cluster will be observable in the pattern. Once the subjects are clustered, data from representative subjects, or average data from the subjects in a single cluster, may be used to develop molecular systems patterns, e.g., images, in which the elements of a pattern represent biomolecules, thereby providing a pattern that is indicative of the biochemical effect of the drug on a subject. The ability to determine which drugs operate on different pathways will be useful in early stage pharmaceutical development, as effort can be concentrated on the best drug in each distinct cluster or class, rather than pursuing a duplicative effort.

### EXAMPLE 10

### Comparison of Toxic Effects of Drugs

Subjects that exhibit the same toxicity phenotype are studied. Data on the concentration of biomolecules are obtained from each subject in the population and on controls. An SOM algorithm is then applied to the data to create a pattern, in which the individual elements represent one or more subjects, as opposed to biomolecules. Distinct clusters of subjects are observable in the pattern for each different type of toxicity regardless of whether the toxicity has observable physiological consequences. For example, liver, kidney, or neurological toxicity may lead to similar phenotypes. Once the subjects are clustered, data from representative subjects, or average data from the subjects in a single cluster, may be used to develop molecular systems images in which the elements of a pattern represent biomolecules, thereby providing a pattern that is indicative of a particular toxic effect in a subject.

### EXAMPLE 11

### MSIs Produced from Rodents

The goal of this example is to demonstrate the power of molecular systems imaging to define a disease phenotype visually. The general area of medical interest was metabolic disease, and the materials to be analyzed were serum samples from a rodent species. Two groups of rodents, diseased and healthy, were employed in the study. A subset of each group was drug treated, yielding the test set:

8 control rodents treated with vehicle,

8 control rodents treated with drug,

8 diseased rodents treated with vehicle, and

8 diseased rodents treated with drug.

Samples were taken from each of the 32 test rodents and analyzed via the lipid LC/MS platform. A molecular systems image map was then trained on this data set to define the spatial location of each of the metabolites on the final image.

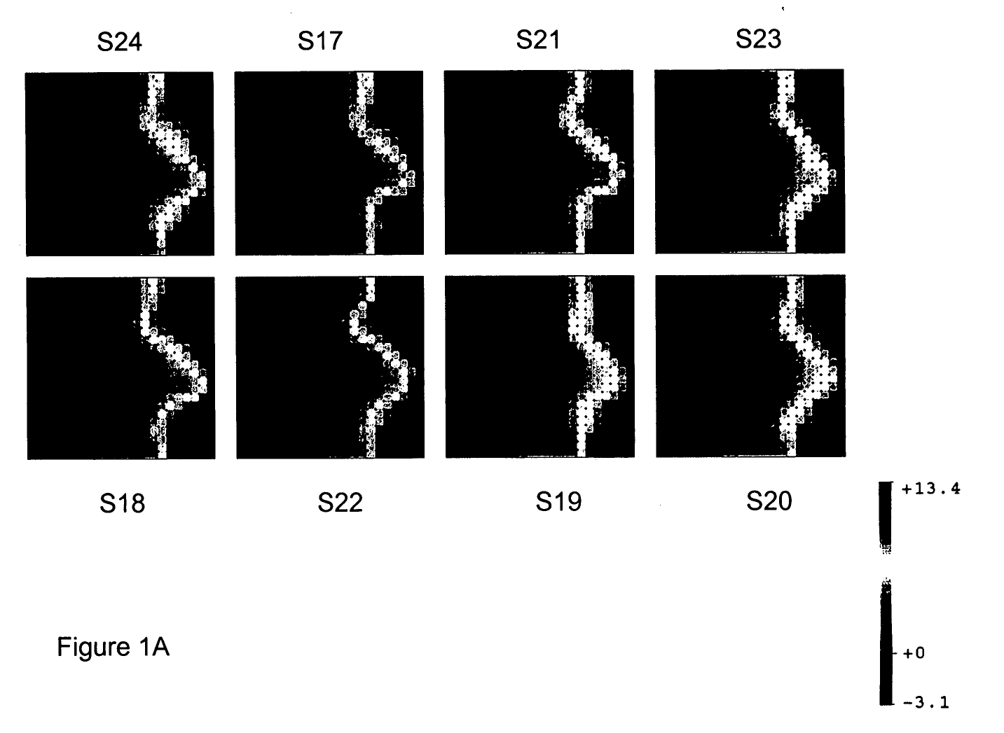

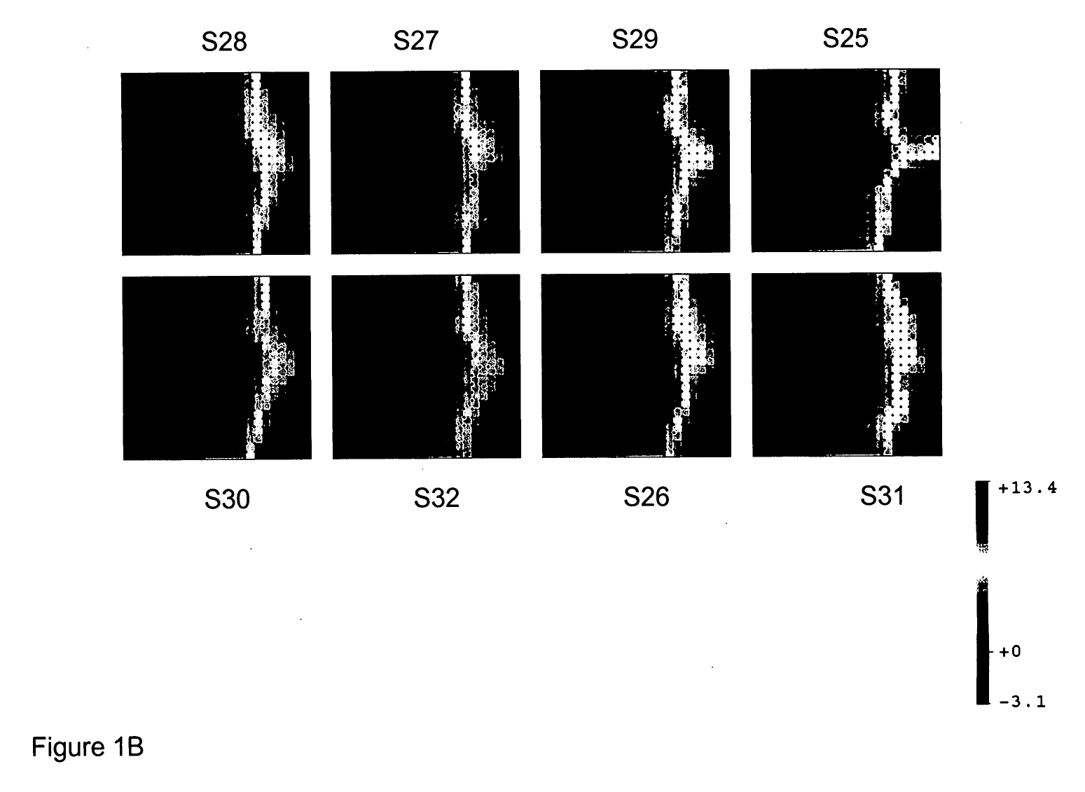

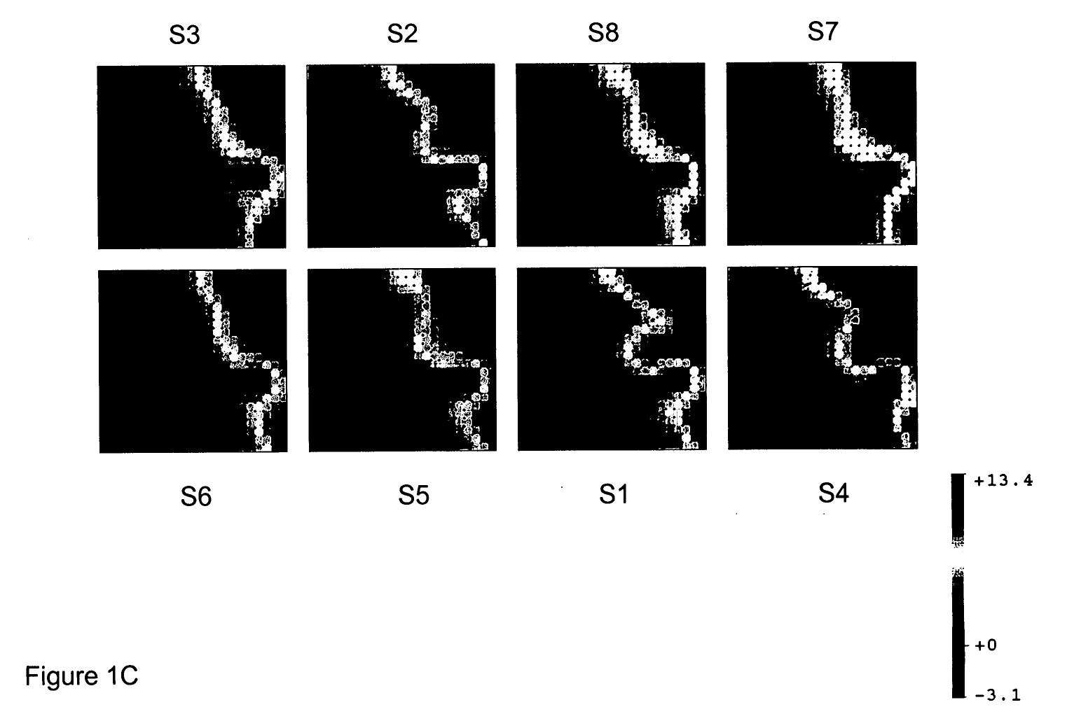

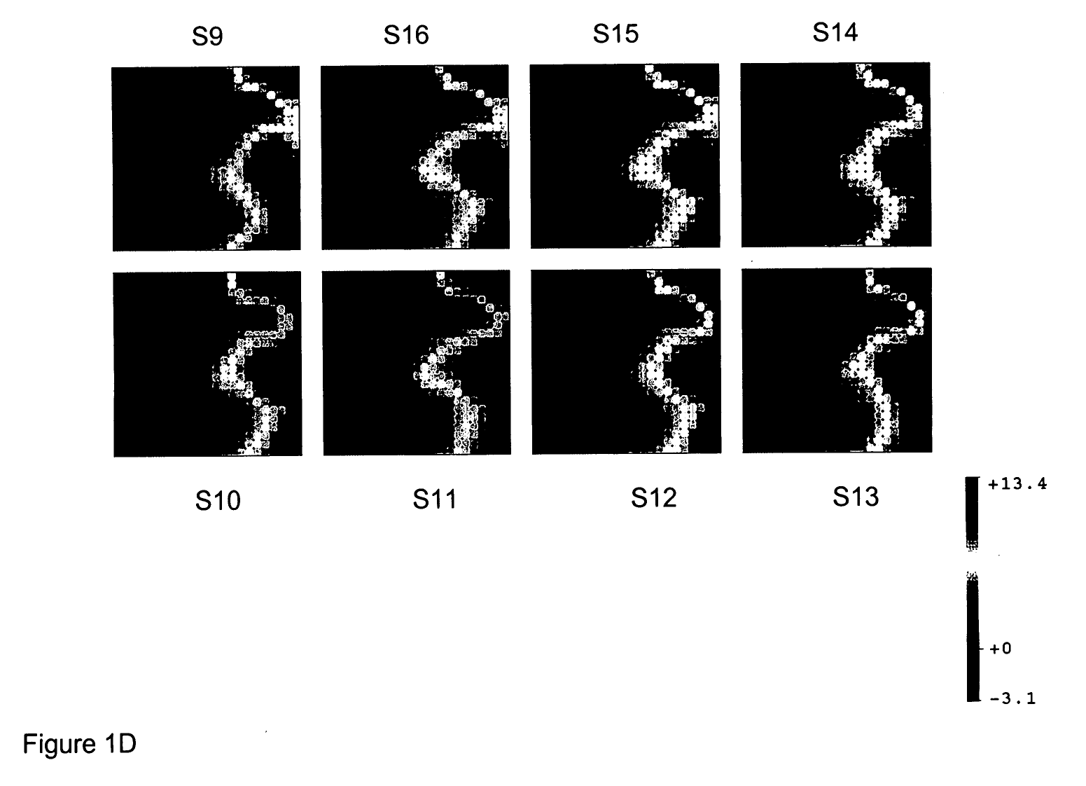

A molecular systems image (MSI) was then constructed for each sample (FIGS. 1A-1D). Each MSI pixel represents zero, one, or multiple metabolite peak(s) from an LC/MS analysis of a sample. The metabolite peak to pixel relationship is determined by a self-organizing map (SOM) algorithm designed to minimize the difference in color between adjacent pixels across all samples. The color of the pixel displayed in each case is the normalized magnitude of that peak in arbitrary units, with red being the highest numerical value and blue being the lowest. FIG. 1A shows MSIs from the eight healthy rodents that had been administered a vehicle. FIG. 1B shows MSIs from the eight healthy rodents that had been administered the drug. FIG. 1C shows MSIs from the eight diseased mammals that had been administered vehicle. FIG. 1D shows MSIs from the eight diseased mammals that had been administered the drug, which was known to treat the disease. Note that the MSIs of the individual rodents in each group can readily be perceived as similar or essentially the same; and that MSIs from the same rodent but in a different biological state can be perceived as different. Note also that the MSIs in FIG. 1A (healthy rodents) are similar to those in FIG. 1D (diseased but drug treated), indicating that the drug likely is therapeutically effective in treating the diseased rodents.

### EXAMPLE 12

### Systems Pathology of a Disease Model

An illustrative example of the techniques of systems pathology were applied to a model of the disease atherosclerosis, the apolipoprotein E3-Leiden (APOE*3-Leiden, APOE*3) transgenic mouse. Apo E is a component of very low density lipoproteins (VLDL) and VLDL remnants and is required for receptor-mediated re-uptake of lipoproteins by the liver. [Glass and Witztum, Cell 104, 502 (1989).] The APOE*3-Leiden mutation is characterized by a tandem duplication of codons 120-126 and is associated with familial dysbetalipoproteinemia in humans. [van den Maagdenberg et al., Biochem. Biophys. Res. Commun. 165, 851 (1986); and Havekes et al., Hum. Genet. 73, 157 (1986).] Transgenic mice over expressing human APOE*3-Leiden are highly susceptible to diet-induced hyperlipoproteinemia and atherosclerosis due to diminished hepatic LDL receptor recognition, but, when fed a normal chow diet, they display only mild type I (macrophage foam cells) and II (fatty streaks with intracellular lipid accumulation) lesions at 9 months. [Jong et al., Arterioscler. Thromb. Vasc. Biol. 16, 934 (1996).]

APOE*3-Leiden transgenic mouse strains were generated by microinjecting a twenty-seven kilobase genomic DNA construct containing the human APOE*3-Leiden gene, the APOC1 gene, and a regulatory element termed the hepatic control region that resides between APOC1 and APOE*3 into male pronuclei of fertilized mouse eggs. The source of eggs was superovulated (C57B1/6J×CBA/J) F1 females. Transgenic founder mice were further bred with C57B1/6J mice to establish transgenic strains. Transgenic and non-transgenic littermates of F21-F22 generations were used in these experiments. All mice were fed a normal chow diet (SRM-A, Hope Farms, Woerden, The Netherlands) and sacrificed at nine weeks, at which time plasma samples were taken and frozen in liquid nitrogen. Lipid differential profiling analysis was then performed on each plasma sample.

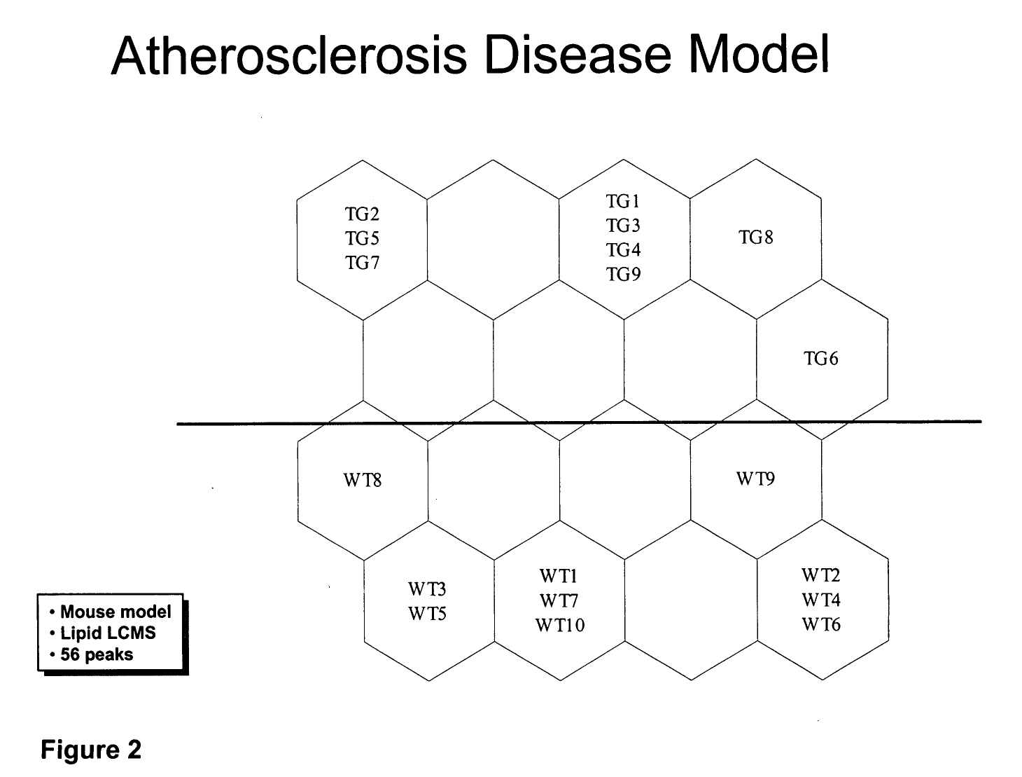

The results of these plasma lipid differential profiling analyses (56 lipid peaks×19 samples) were then used to produce a molecular pathology map for atherosclerosis (FIG. 2). The molecular pathology map separates the transgenic mice from the wild type mice in an unsupervised manner.

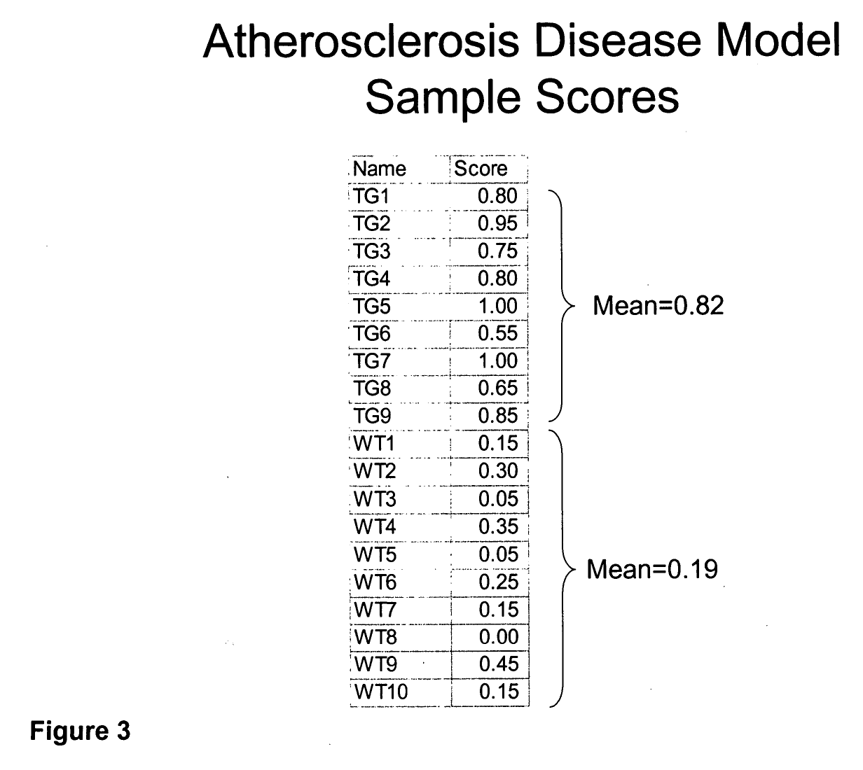

The same set of lipid data was then used to create a 1-D numerical pathology score for each of the samples. The purpose of the pathology score is to classify each sample as either diseased or normal. The score was computed by constructing a 1-D self-organizing map of the sample data. There are other methods of constructing such a score known to those skilled in the art, such as a principle component projection, linear classifier, or nonlinear classifier. In the present case, taking the axis of the self-organizing map as running from left to right, the score was computed as the horizontal position of each sample on the trained map, and normalizing these positions to be between 0 (left-most) and 1 (right-most). The scores are shown in FIG. 3. The maximum score for a wild type (WT) sample is 0.45, and the minimum score for a transgenic (TG) sample is 0.55, indicating that scoring metric can distinguish between diseased and normal.

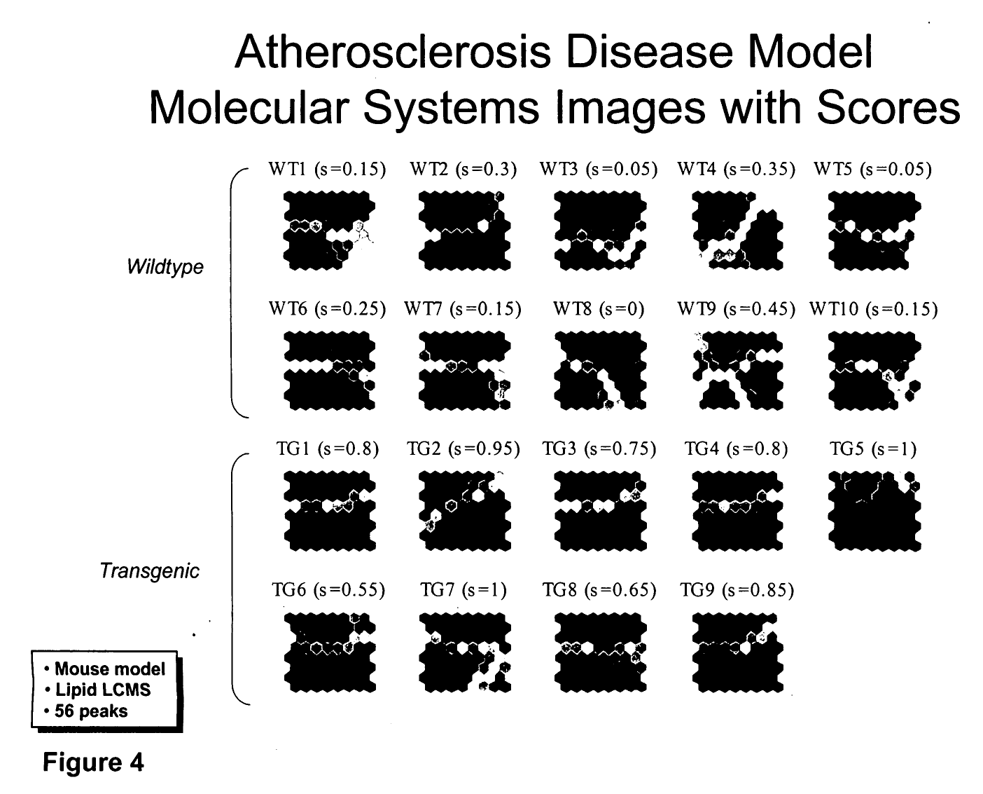

The same set of lipid data was then used to train a molecular systems image map. This map defined the spatial location of each of the metabolites on the final image. A molecular systems image (MSI) was then constructed for each sample (FIG. 4). As in FIG. 1, each MSI pixel represents zero, one, or multiple metabolite peak(s) from an LC/MS analysis of a sample. The color of the pixel displayed in each case is the normalized magnitude of that peak in arbitrary units, with red being the highest numerical value and blue being the lowest.

### OTHER EMBODIMENTS

Each of the patent documents and scientific publications disclosed herein is incorporated by reference herein for all purposes.

Although the invention has been particularly shown and described with reference to specific embodiments, it should be understood by those skilled in the art that various changes in form and detail may be made therein without departing from the spirit, essential characteristics or scope of the invention. The foregoing embodiments are therefore to be considered in all respects illustrative rather than limiting on the invention described herein. The scope of the invention is thus indicated by the appended claims rather than by the foregoing description, and all changes which come within the meaning and range of equivalency of the claims are therefore intended to be embraced therein.

Other embodiments are in the claims.

What is claimed is:

1. A first molecular systems image characteristic of a biological state of a first individual mammal, the image comprising a multidimensional array of data points representative of the relative concentrations of a multiplicity of biomolecules detected in a sample from said mammal in said biological state, the data points being positioned using a mapping key to produce an image which is recognizable by human vision as being distinct from an image generated from a comparable sample from a mammal of the same species in a different biological state.

2. A set of molecular systems images comprising at least:
a) the first molecular systems image of claim 1, and
b) a second, reference image for visual comparison with the image of claim 1, said reference image having been generated by the method and detecting the same or homologous biomolecules used to generate the image of claim 1, except that each data point in the reference image represents one or more biomolecules sampled from a mammal in a known biological state.

3. The set of images of claim 2, wherein the reference image is generated from multiple mammals of the same species as the mammal used to generate the first image.

4. The set of images of claim 3, wherein the mammals used to generate the reference image were, prior to samples having been taken from them, determined not to have a particular disease state, and the mammal used to generate the first image is suspected of having said particular disease state.

5. The set of images of claim 3, wherein the mammals used to generate the reference image were, prior to samples having been taken from them, determined to have a particular medical condition, and the mammal used to generate the first image is suspected of having said particular medical condition.

6. The set of images of claim 3, wherein the mammals used to generate the reference image was, prior to samples having been taken from it, determined not to have been administered a particular drug, and the first mammal used to generate the first image was, prior to a sample having been taken from it, administered said particular drug.

7. The set of images of claim 3, wherein the mammals used to generate the reference image were, prior to samples having been taken from it, administered a particular drug, and the first mammal used to generate the first image was, prior to a sample having been taken from it, administered said particular drug.

8. The set of images of claim 3, further comprising a third molecular systems image generated from a second individual mammal of the same species as the first mammal, said third image having been generated by the method, and detecting the same biomolecules used to generate the image from the first mammal, except that the third mammal is in a different biological state from the first mammal.

9. The set of images of claim 3, further comprising a third molecular systems image generated from said first individual mammal by the method, and detecting the same biomolecules used to generate the first image, except that the third image is generated using a sample taken from the mammal at a different point in time from the point in time of the taking of the sample used to generate the first image.

10. The image of claim 1, wherein the image comprises an array of pixels arranged in a cluster-based pattern wherein the pixels in the array can vary from other pixels in the array in shape, color, or shade to indicate biomolecule concentration.

11. The image of claim 1, wherein the mapping key is generated by a self-organizing map algorithm operating on a study data set.

12. The image of claim 1, wherein the biological state is normal, homeostatic, diseased, environmentally, physically or mentally stressed, intoxicated, successfully or unsuccessfully drugged, aged, embryonic, nutrient deprived, obese, hungry, or thirsty.

13. The image of claim 1, wherein the mammal is a human.

14. The image of claim 1, wherein the mammal is an experimental animal.

15. The image of claim 14, wherein the experimental animal is a genetically altered animal.

16. The image of claim 1, wherein said sample is a liquefied tissue sample, whole blood, a blood fraction, urine, saliva, lymph, cerebrospinal fluid, mucous, nipple secretion, feces, ocular fluid, or a combination thereof.

17. The image of claim 1, wherein the biomolecules comprise at least one lipid.

18. The image of claim 17, wherein the biomolecules comprise multiple different lipids.

19. The image of claim 18, wherein the biomolecules comprise more than 10 different lipids.

20. The image of claim 17, wherein the biological state is metabolic disorder.

21. The image of claim 1, wherein the biomolecules comprise at least two of proteins, peptides, lipids, and metabolites.

22. The image of claim 21, wherein the biomolecules comprise mRNA.

23. The method of claim 1, wherein biomolecules are detected using one or more of the techniques of mass spectrometry, liquid chromatography, gas chromatography, and nuclear magnetic resonance spectroscopy.

24. A method for assessing the toxicity of a substance, said method comprising the steps of:
a) providing a first, test molecular systems pattern comprising a multiplicity of data points representative of the relative concentrations of a multiplicity of biomolecules detected in a sample from a test mammal to which the substance has been administered, the data points being clustered to produce said pattern which is recognizable by a computer or by human vision,
b) providing a second, reference molecular systems pattern generated by the method and detecting the same biomolecules used to generate the first pattern, except that the sample(s) used to generate the reference pattern are obtained from a different mammal or multiple mammals of the same species as the first mammal, and
c) comparing the first pattern with the second, reference pattern.

25. The method of claim 24, further comprising the step, if the comparison indicates possible toxicity, of comparing the first pattern to one or more third patterns generated by the method and detecting the same biomolecules used to generate the first pattern, said one or more third patterns having been generated using samples from mammals known to have been exposed to or administered a toxic substance, wherein a substantial similarity of said first pattern and a said third pattern is indicative of probable toxicity.

26. A method for assessing the toxicity of a substance, the method comprising the steps of:
a) providing a test molecular systems pattern comprising a multiplicity of data points representative of the relative concentrations of a multiplicity of biomolecules detected in a sample from a first mammal to which the substance has been administered, the data points being clustered to produce said pattern which is recognizable by a computer or by human vision,
b) providing one or more second, reference molecular systems patterns generated by the method and detecting the same biomolecules used to generate the first pattern, except that the samples used to generate the reference patterns are obtained from a different individual or multiple individuals of the same species as the first mammal, which individuals have not been exposed to or administered the substance, and which have been treated with a different substance known to be toxic to mammals of said species, and
c) comparing the first and second molecular systems patterns, a substantial similarity of the first pattern with a said second pattern being indicative of probable toxicity.

27. A method for assessing the efficacy of a drug candidate for treating a disease state, said method comprising the steps of:
a) providing a first molecular systems pattern comprising a multiplicity of data points representative of the relative concentrations of a multiplicity of biomolecules detected in a sample from a first mammal having a disease state to which the drug candidate has been administered, the data points being clustered to produce said pattern which is recognizable by a computer or by human vision,
b) providing one or more second, reference molecular systems patterns generated by the method and detecting the same or homologous biomolecules used to generate the first pattern, except that the sample(s) used to generate the reference patterns are obtained from a different individual or multiple individuals of the same species as the first mammal, to which the drug candidate has not been administered and which do not have the disease state or have been effectively treated for the disease state, and
c) comparing the first and second molecular systems patterns, a substantial similarity of the first pattern with a said second pattern being indicative of probable efficacy.

28. The method of claim 27, wherein the drug candidate comprises a combination of two or more biologically active substances.

29. The method of claim 28, wherein at least one of the substances in the combination is, prior to administration to the mammal, known to have efficacy in treating the disease state.

30. The method of claim 28, wherein at least one of the substances in the combination is, prior to administration to the mammal, designed by a rational drug design method aimed at the disease state.

31. A method for generally determining whether a human subject is in a disease state, said method comprising the steps of:
a) providing a first molecular systems pattern comprising a multiplicity of data points representative of the relative concentrations of a multiplicity of biomolecules detected in a sample from the subject, the data points being clustered to produce said pattern which is recognizable by a computer or by human vision;
b) providing one or more second, reference molecular systems patterns generated by the method and detecting the same biomolecules used to generate the first pattern, provided that the sample(s) used to generate the reference patterns are obtained from a different human subject or subjects known not to be in disease states; and
c) comparing the first and second molecular systems patterns, a substantial difference in patterns being indicative of a probable disease state in the first subject.

32. A method for determining the likely presence of a particular disease state in a human subject, said method comprising the steps of:
a) providing a first molecular systems pattern comprising a multiplicity of data points representative of the relative concentration of a multiplicity of biomolecules detected in a sample from the subject, the data points being clustered to produce said pattern which is recognizable by a computer or by human vision;
b) providing one or more second, reference molecular systems patterns generated by the method and detecting the same biomolecules used to generate the first pattern, provided that the sample(s) used to generate the reference patterns are obtained from a different human subject or subjects known to be in said disease state; and
c) comparing the first and second molecular systems patterns, a substantial similarity in patterns being indicative of said probable disease state in the subject.

33. A method for monitoring the course of a particular disease state in a human patient known to have said disease, said method comprising the steps of:
a) providing two or more molecular systems patterns, each comprising a multiplicity of data points representative of the relative concentrations of a multiplicity of biomolecules detected in two or more samples taken from the patient at different points in time, the data points being clustered to produce, for each sample, said pattern which is recognizable by a computer or by human vision; and
b) comparing the two or more molecular systems patterns, substantial changes in the patterns over time being indicative of a change in the disease state.

34. The method of any one of claims 24-33, wherein the molecular systems patterns are images recognizable by human vision.

35. A molecular pathology map which represents biochemical variation in multiple mammals of the same species, all of which exhibit similar negative or positive phenotype with respect to a particular disease state, said map comprising a multi-dimensional array of data points, wherein:
a) each data point represents a composite value, for one of said multiple mammals, of the relative concentrations of multiple biomolecules detected in a sample from the mammal, the composite value having been derived in the same manner for each mammal, and
b) the data points in the array are clustered by an algorithm that groups individual mammals according to similarity of composite values for concentrations of said biomolecules.

36. The map of claim 35, wherein:
i) the mammals all exhibit a particular disease state,
ii) the sample type taken from each animal is relevant to the disease state, and
iii) at least some of the biomolecules detected in the samples are relevant to the disease state.

37. The map of claim 35, wherein the mammals are humans.

38. The map of claim 35, wherein the mammals are non-human experimental animals.

39. The map of claim 36, wherein different clusters of mammals on the map are representative of different sub-types of said disease state.

40. The map of claim 35 further comprising links at points thereon to underlying data supporting said points which permit an investigator to explore the biochemistry of individual said mammals.

41. A method of obtaining information about sub-types of a particular disease state, said method comprising the steps of:
a) providing a molecular pathology map of claim 35 for said disease state, and
b) comparing the biochemistry of individuals within clusters of said map to biochemistry data relevant to said disease state.

42. A method of biochemically categorizing human subjects who have been administered the same biologically active substance, wherein the subjects exhibit a negative or positive phenotype with respect to a disease state, said method comprising the steps of:
a) providing a molecular pathology map of claim 35 for the subjects, and
b) ascertaining clustering patterns within the map, such patterns indicating different physiological responses to said biologically active substance.

43. The method of claim 42, wherein the subjects comprise two groups which phenotypically respond differently from each other to said biologically active substance.

44. The method of claim 43, wherein said phenotypic response is mitigation or prevention of the disease state.

45. The method of claim 43, wherein said phenotypic response is a deleterious side effect of said biologically active substance.

46. The method of claim 45, wherein the map is compared to a composite value data point, as defined in claim 35, for an individual human subject to whom said biologically active substance has been administered, said data point having been generated by the same method, and detecting the same biomolecules, as used to generate the data points of the maps.

47. The method of claim 46, wherein mapping of said individual data point more closely to a group responding deleteriously to the biologically active substance disqualifies the individual from treatment of the disease state with the biologically active substance.

48. The method of claim 24, wherein the mammals used to generate the reference pattern have been administered the substance, in the same manner as the test mammal.

49. The method of claim 48, wherein some of the reference mammals exhibited, prior to generation of the reference pattern, a side effect in response to the substance, and some of the reference mammals did not, prior to generation of the reference pattern, exhibit a side effect in response to the substance, and wherein the side effect group exhibits a different pattern from the no side effect group in the reference pattern.

50. The method of claim 49, wherein the comparison of patterns is carried out in connection with a planned or ongoing clinical trial of the substance, and the mammals are human subjects.

51. The method of claim 50, wherein the human subjects used to generate the test and reference molecular systems patterns have the same disease state, and the substance is a drug candidate for mitigating or preventing said disease state.

52. The method of claim 51, wherein, if the pattern for the test subject is more similar to the side effect reference pattern, the subject is excluded from the clinical trial.

53. A method for assessing the potential of a human subject with a disease state for suffering a side effect from a drug candidate for treating said disease state, said method comprising the steps of:
a) providing a first, test molecular systems pattern comprising a multiplicity of data points representative of the relative concentrations of a multiplicity of biomolecules detected in a sample from said test human subject to which the drug candidate has not been administered, the data points being clustered to produce said pattern which is recognizable by a computer or by human vision,
b) providing one or more second, reference molecular systems patterns generated by the method and detecting the same biomolecules used to generate the test pattern, except that the sample(s) used to generate the reference patterns are obtained from multiple human subjects to whom the drug candidate has been administered, wherein a first sub-group of the reference subjects suffered a side effect from the drug candidate and a second subgroup did not, and
c) comparing the first, test pattern with the one or more second reference patterns.

54. The method of claim 53, wherein the comparison of patterns is carried out in connection with a planned or ongoing clinical trial of the drug candidate, and a test subject with a test pattern similar to the side effect sub-group is excluded from the clinical trial.

55. A method for obtaining information about the biological state of a test human subject, said method comprising the steps of:
a) administering to said subject, in a sub-toxic dose either a drug, or a biologically active surrogate substance,
b) obtaining a sample from said subject,
c) generating, from said sample, a molecular systems test pattern comprising a multidimensional array of data points representative of the relative concentrations of a multiplicity of biomolecules detected in the sample, the data points being clustered to produce a pattern which is recognizable by a computer or human vision,
d) providing a first composite reference pattern generated by the method of steps a-c) and detecting the same biomolecules used to generate the pattern of step c), except that each data point in the first composite reference pattern represents a composite of samples from multiple human subjects who have responded to an efficacious dose of the drug in a clinically acceptable manner,
e) providing a second composite reference pattern generated by the method of step d) except that the samples used to generate the patterns are obtained from subjects who have responded to the drug in a clinically unacceptable manner, and
f) comparing the test pattern of step c) with the reference patterns of steps d) and e) to predict the biological state of said subject.

56. The method of claim 55, wherein said biological state is the potential for said test human subject with a disease state to experience a benefit or a deleterious side effect from the administration of a drug, said method serving to predict the response of the test subject to an efficacious dose of the drug.

57. A method of differentiating the biochemical toxicity pathways for two drugs that cause toxicity in the same organ or tissue, said method comprising the steps of:
a) administering each drug to a group of human subjects,
b) obtaining from each said subject a sample relevant to the tissue or organ to which the drug is toxic,
c) generating, from the samples in each of the two groups, a composite reference pattern comprising a multidimensional array of composite data points, each representing a composite of data from samples from the group, the data from each sample representing the relative concentrations of a multiplicity of biomolecules, wherein the composite data points of the array for each group are clustered by an algorithm to produce said pattern which is recognizable by a computer or by human vision, and
d) comparing the composite patterns for each group to elucidate different toxicity pathways.

58. A method for assessing the toxicity of a substance, the method comprising the steps of:
a) providing a test molecular systems pattern comprising a multiplicity of data points representative of biological measures detected in a sample from a first mammal to which the substance has been administered, the data points being clustered to produce said pattern which is recognizable by a computer or by human vision,
b) providing one or more second, reference molecular systems patterns generated by the method and detecting the same biological measures used to generate the first pattern, except that the samples used to generate the reference patterns are obtained from a different individual or multiple individuals of the same species as the first mammal, which individuals have not been exposed to or administered the substance, and which have been treated with a different substance known to be toxic to mammals of said species, and
c) comparing the first and second molecular systems patterns, a substantial similarity of the first pattern with a said second pattern being indicative of probable toxicity.

59. A method for assessing the efficacy of a drug candidate for treating a disease state, said method comprising the steps of:
a) providing a first molecular systems pattern comprising a multiplicity of data points representative of biological measures detected in a sample from a first mammal having a disease state to which the drug candidate has been administered, the data points being clustered to produce a pattern which is recognizable by a computer or by human vision,
b) providing one or more second, reference molecular systems patterns generated by the method and detecting the same or homologous biological measures used to generate the first pattern, except that the sample(s) used to generate the reference patterns are obtained from a different individual or multiple individuals of the same species as the first mammal, to which the drug candidate has not been administered and which do not have the disease state or have been effectively treated for the disease state, and
c) comparing the first and second molecular systems patterns, a substantial similarity of the first pattern with a said second pattern being indicative of probable efficacy.
